(19) United States
(12) Patent Application Publication
(10) Pub. No.: US 2017/0119937 A1
(43) Pub. Date: May 4, 2017

(54) SURGICAL SUCTION DEVICE

(71) Applicants: Zohny Zohny, St. Louis, MO (US); Junwoo Suh, St. Louis, MO (US); Suh Woo Jung, St. Louis, MO (US)

(72) Inventors: Zohny Zohny, St. Louis, MO (US); Junwoo Suh, St. Louis, MO (US); Suh Woo Jung, St. Louis, MO (US)

(21) Appl. No.: 15/337,570

(22) Filed: Oct. 28, 2016

Related U.S. Application Data
(60) Provisional application No. 62/285,418, filed on Oct. 29, 2015.

(51) Int. Cl. A61M 1/00

(52) U.S. Cl. CPC A61M 1/0023; A61M 1/0094; A61M 2202/0413

(57) ABSTRACT

A surgical suction device includes a vacuum source, hose member, vacuum adaptor, and suction attachment. The suction attachment includes each of a bottom layer, middle layer, and a top layer. The bottom layer and the top layer are made of an absorptive material. The middle layer includes a system of microtubules that are in fluid communication with the vacuum source via several apertures located on its top surface. When the device is in use, the bottom layer is in contact with the soft tissue that is being operated on. When the vacuum source is turned on, suction is provided through the vacuum apertures on top of the middle layer via the microtubules. The suction persists through the top layer, and any fluid in the surgical site above the top layer is sucked through the top layer into the vacuum apertures, through the microtubules, and out of the suction attachment.

### CROSS-REFERENCE TO RELATED APPLICATIONS

This application claims the benefit of U.S. Provisional Application No. 62/285,418, filed Oct. 29, 2015, which is hereby incorporated by reference in its entirety.

### FIELD OF THE INVENTION

The present invention relates to absorptive surgical strips and a suction device. The strips and suction device are used during a surgery, such as a craniotomy, to vacuum and absorb excess blood or other fluid oozing from the walls of a cavity and to create physical barriers between healthy brain tissues and tumor tissues.

### BACKGROUND OF INVENTION

During neurosurgery and other intricate surgical procedures, blood and other bodily fluid often pool at the surgical site. The pooling can lead to serious complications. Thus, several commercially available products seek to offer convenient and fast means of preventing blood pooling in surgical cavities. However, lack of a fluid control system in the cavity leads to quick saturation of absorptive surgical strips such as cotton “patties.” As the patties are saturated, they must be constantly replaced, and the surgical site must also be regularly irrigated with a sterile solution. These processes complicate a surgical procedure and further can damage soft tissues due to the adhesive quality of the cotton patties.

American Surgical Company, a manufacturer of the most commonly used cotton patties in the United States, seeks to mitigate the brain tissue damage by developing versatile neurosurgical patties such as Ray-Cot®, Delicot®, and TELFA®. These newly developed cotton patties are coated with different types of biocompatible polymers that are less adhesive so as to reduce the likelihood of the patties sticking to soft tissue. Yet, studies continue to address concerns that these modifications do not significantly reduce the damage to soft tissue, especially when the patties are wet. Moreover, despite the constant effort to increase the absorbency of cotton patties, their capacities are strictly limited to their material properties, and they are far from overcoming the need for active removal (i.e. suction/aspiration).

In addition to creating the need for the frequent replacements of surgical patties and the application of irrigation fluid, the excess blood oozing creates more inefficiency by filling up the cavity and thus obscuring the surgical theatre. In order to maintain operative visibility, a neurosurgeon must constantly use manual suction techniques, frequently replace cotton patties, and apply irrigation fluid. These tasks greatly disrupt a surgeon's workflow, thus increasing the likelihood of leaving a greater post-surgery trauma on a patient's soft tissues. Therefore, there is a strong clinical need for a more efficient fluid control system in the cavities such that blood pooling is reduced effectively and safely.

### SUMMARY OF INVENTION

An improved surgical suction device is provided that includes each of a vacuum source, a hose member, vacuum adaptor, and a suction attachment. The suction attachment generally includes each of a bottom layer, middle layer, and a top layer. Both of the bottom layer and the top layer are preferably comprised of cotton, but in alternative embodiments may be made up of another absorptive material. The middle layer is preferably made of a hydrophobic Polydimethylsiloxane (PDMS).

When the surgical suction device is in use, the bottom layer is in contact with the soft tissue that is being operated on, for example brain tissue during neurosurgery. The top layer and the middle layer are positioned and located distal to the surgical site during an operation. When a procedure is underway, the bottom layer acts as an absorptive layer able to absorb and retain excess fluid such as blood that pools at the surgical site. When the vacuum source is turned on, it is able to provide suction via a hose member and a vacuum adaptor in fluid connection with the suction attachment through several vacuum apertures that are placed on the upper surface area of the middle layer.

The various vacuum apertures and the middle layer are interconnected to one another, as well as the vacuum adaptor, via a plurality of microtubules. When the vacuum source is turned on, suction is provided through the vacuum apertures via the microtubule system. The suction persists through the top layer, such that any fluid in the surgical site that flows up above the top surface of the top layer is sucked through the top layer into the vacuum apertures, through the microtubules, and out of the suction attachment via the vacuum adaptor, hose member, and into the vacuum source or other waste container. At the same time, the top layer acts as an absorptive layer that helps to absorb excess blood and other fluid that is pooling in the surgical site.

A number of configurations for the vacuum apertures and the microtubules are available. In the preferred embodiment, there are five vacuum apertures, which are interconnected by the plurality of microtubules. In any embodiment, the microtubules are in fluid communication with the vacuum adaptor. Because the suction device includes each of a vacuum source and top and bottom absorptive layers, the surgical suction device acts as both a passive and active fluid absorbent device. The device eliminates the need for a surgeon or medical assistant to use one hand to absorb excess pooling blood and the other hand to maneuver a suction device and/or apply irrigation fluid during an operation.

### BRIEF DESCRIPTION OF DRAWINGS

FIG. 1 is a perspective view of a surgical suction device constructed according to the teachings of the invention;

FIG. 2 is an exploded plan view of the suction device of FIG. 1;

FIG. 3 is a cross-section view of the middle layer of FIG. 2;

FIG. 4 is a top plan view of an alternative middle layer to the middle layer shown in FIG. 2; and

FIG. 5 is a cross-section view of the alternative chip member of FIG. 4.

### DETAILED DESCRIPTION OF THE INVENTION

A surgical suction device 1 is provided that allows a neurosurgeon or other medical professional to absorb excess blood that is oozing from the walls from a cavity within a surgical site. The suction device 1 preferably includes a vacuum source 3 that is of the type known and understood in the art. The vacuum source 3 may be powered by batteries, but in the preferred embodiment it is powered by plugging a power cord of the vacuum source 3 into a wall. The vacuum source 3 is also in fluid communication with a hose member 5. The hose member 5 transports fluid that the suction device 1 is able to suck up from the surgical site to the vacuum source 3. A vacuum adaptor 7 is also provided that is in fluid communication with, and is releasably attachable to, a suction attachment 9. As discussed in greater detail below, when the vacuum source 3 is activated, any excess fluid that has accumulated in the surgical site is vacuumed through the suction attachment 9, the adaptor 7, the hose member 5, and out through the vacuum source 3.

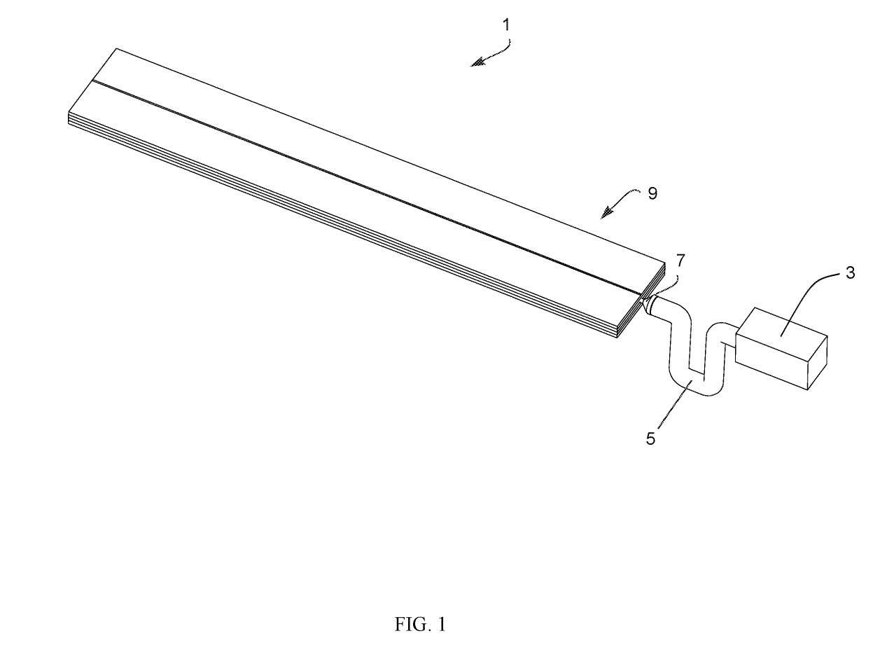

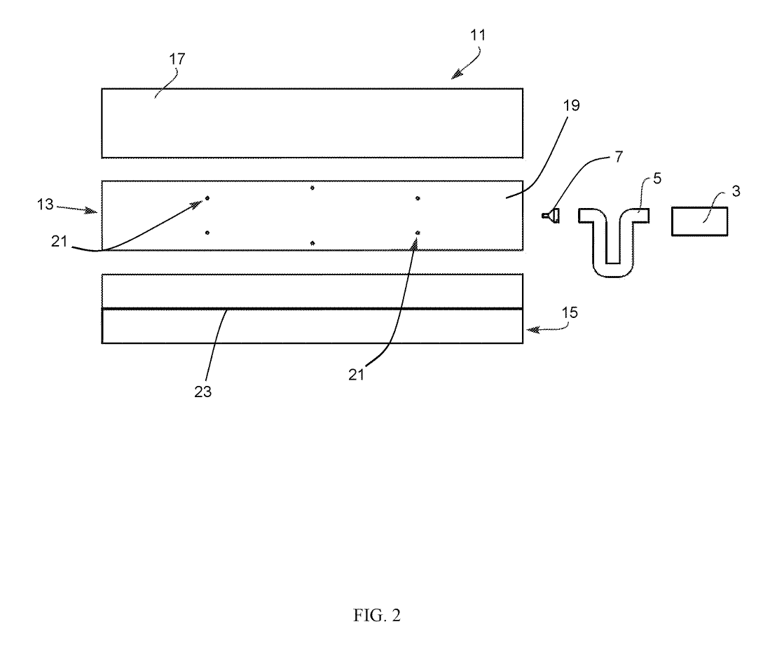

As provided in FIG. 2, the suction attachment 9 includes each of a bottom layer 11, middle layer 13 and a top layer 15. The bottom layer 11 is preferably constructed of cotton and may be sized and shaped as substantially shown in FIG. 2. The bottom layer 11 includes each of an adhesion side 17 and a surgical side (not illustrated). The surgical side is the side of the bottom layer 11 that is adjacent the soft tissue during a surgical procedure. The surgical side must be constantly wetted or irrigated during the procedure so as to reduce the likelihood that the bottom layer 11 will stick to the soft tissue, and thus damage the tissue when the bottom layer 11 and the suction attachment 9 are removed following the completion of a procedure. The adhesion side 17 is adhered to the middle layer 13 when the suction attachment 9 is assembled (as illustrated in FIG. 1).

The middle layer 13 may be adhered to the adhesion side 17 of the bottom layer 11 by traditional adhesive substances so long as the adhesive substances do not cause adverse biological effects. The bottom layer 11 is adhered to the middle layer 13 at a bottom side (not illustrated) of the middle layer 13. By being adhered to the middle layer 13, the bottom layer 11 is in direct contact with the soft tissue during a procedure, not the middle layer 13. A top side 19 of the middle layer 13 is adhered to the top layer 15 using a known biocompatible adhesion method.

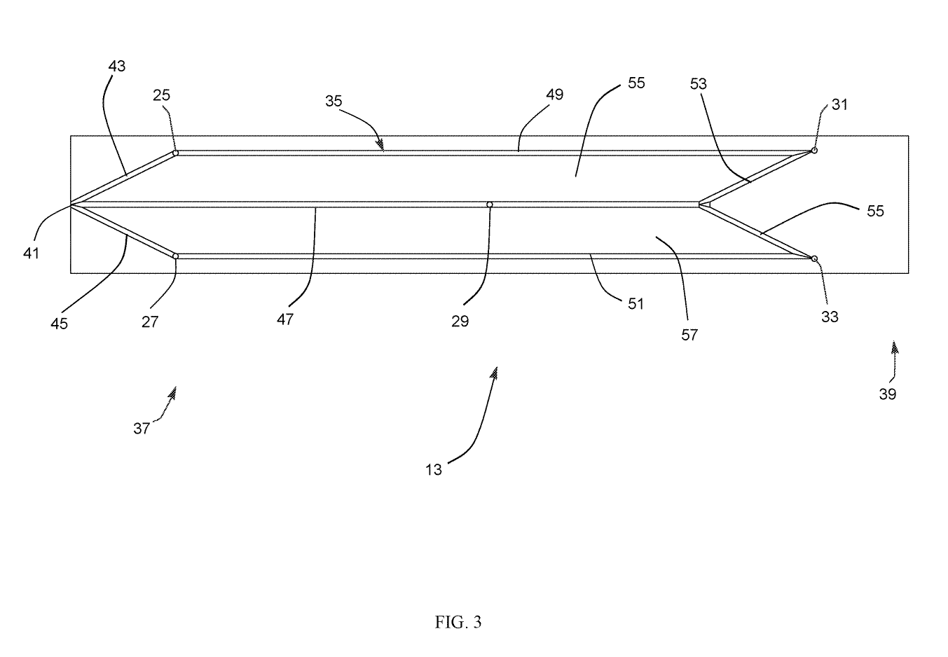

The middle layer 13 shown in FIG. 2 includes a plurality of vacuum apertures 21. The apertures 21 are in fluid communication with a microtubule system described below in the description of FIG. 3. The middle layer 13 is preferably made of a hydrophobic Polydimethylsiloxane (PDMS) material. The middle layer 13 may be manufactured using a sugar mold process, as known and understood in the art. The hydrophobicity of the middle layer 13 helps to allow excess fluid to flow through its microtubule system without pooling and clotting therein.

The top layer 15 also includes a bottom side (not illustrated) to which the top side 19 of the middle layer 13 is adhered. As was the case for adhering the bottom layer 11 and the middle layer 13 to one another, the middle layer 13 and the top layer 15 may be adhered to one another using any sort of biocompatible adhesive. By separating the bottom layer 11 from the top layer 15, no fluid exchange can take place between the bottom layer 11 and the top layer 15 during the suction process, as discussed below.

The top layer 15 is also preferably constructed of cotton, though it may be constructed of any suitable material that has absorptive qualities. By covering the middle layer 13 and its vacuum apertures 21 with an absorptive top layer such as top layer 15, the middle layer 13 will likewise not directly contact soft tissue. The top layer 15 shown and illustrated further includes a radioactive tag 23. The radioactive tag 23 acts as a marker that can be detected by a surgeon prior to closing the surgical site so that the top layer 15 or any other part of the suction attachment 9 is not left behind in the surgical site after a procedure is completed.

Turning now to FIG. 3, a cross section view of the middle layer 13 is provided. The middle layer 13, as described above, includes a plurality of vacuum apertures 21. As shown in FIGS. 2 and 3, the middle layer 13 includes five vacuum apertures 21. The middle layer 17 includes an upper left vacuum aperture 25, a lower left vacuum aperture 27, a central aperture 29, an upper right vacuum aperture 31, and a lower right vacuum aperture 33. The apertures 21 do not tunnel entirely through the height of the middle layer 13. Instead, they are sized and structured similarly to cavities that have a depth less than the height of the middle layer 13. That way, the apertures 21 do not apply any suction through the bottom layer 11, and do not introduce negative pressure to the bottom of the device 1, thus preventing suction from being applied directly to soft tissue.

The vacuum apertures 21 are connected to one another via a microtubule system 35 through which excess fluid at a surgical site may be vacuumed. The microtubules described below that make up the microtubule system 35 preferably have a diameter of 1 mm and have a wall thickness of approximately 2 mm, though they could be smaller or larger in alternative embodiments. In some embodiments, to reduce clotting therein, the microtubules of the microtubule system 35 may be lined with heparin or a different blood thinning substance.

The middle layer 13 generally comprises each of a left portion 37 and a right portion 39. At the left portion 37, the middle layer includes an outlet 41 in communication with the microtubule system 35. The outlet 41 is in fluid communication with the vacuum adaptor 7, the hose member 5, and the vacuum source 3 (as illustrated in FIG. 1). The outlet 41 is in fluid communication with the upper left vacuum aperture 25 via a microtubule segment 43. The outlet 41 is also in fluid communication with the lower left vacuum aperture 27 via a microtubule segment 45. A central microtubule segment 47 connects the outlet 41 to the central aperture 29. The upper left vacuum aperture 25 and lower left vacuum aperture 27 are in fluid communication with the upper right and lower right apertures 31, 33, respectively via microtubule segments 49, 51, respectively. The central microtubule segment 47 extends through the central aperture 29, and it is in fluid communication with the apertures 31, 33 at the right side 39 via microtubule segments 53, 55, respectively.

In operation, when the vacuum source 3 is activated, suction is provided at the outlet 41 that causes the various vacuum apertures 21 to suck through the top layer 19 (not illustrated in FIG. 3) any fluid that is above the suction attachment 9. The several vacuum apertures 21 create multiple points of aspiration during suction when the vacuum source 3 is activated, thus helping to ensure that suction is applied even if one of the apertures 21 is blocked by a foreign material. Via the system of microtubules 35, excess fluid around the suction attachment 9 is sucked through the top layer 19, into the various vacuum apertures 21 and through the outlet 41 to the vacuum source 3, where it is disposed of.

The microtubule system 35 and the vacuum apertures 21 in the arrangement shown in FIG. 3 create a pattern that resembles a first parallelogram 55 and a second parallelogram 57, wherein the second parallelogram 57 is a mirror image of the first parallelogram 55.

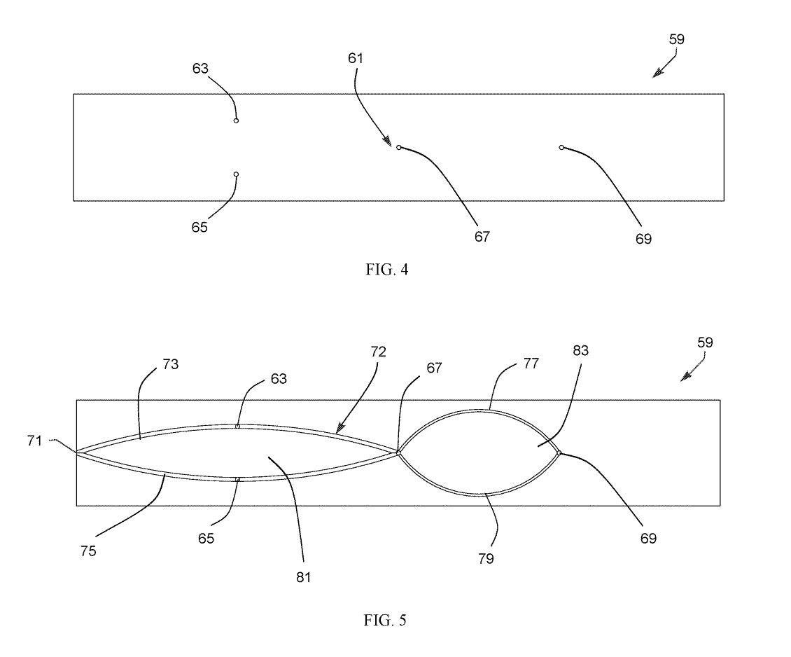

An alternative embodiment of the middle layer 13, middle layer 59, is provided in FIGS. 4 and 5. The middle layer 59 is substantially similar to the middle layer 13 in its size, material composition, and adhesion to the bottom layer 11 and top layer 15. However, middle layer 59 includes vacuum apertures 61 arranged differently than the vacuum apertures 21. As shown in FIGS. 4 and 5, the middle layer 59 includes an upper left vacuum aperture 63, a lower left vacuum aperture 65, a central aperture 67, and a right central aperture 69.

The middle layer 59, like middle layer 13, includes an outlet 71. Like the outlet 41, the outlet 71 is in fluid communication with the vacuum adaptor 7, the hose member 5, and the vacuum source 3 (as illustrated in FIG. 1) via a microtubule system 72 made up of a plurality of microtubule segments. The outlet 71 is in fluid communication with the upper left vacuum aperture 63 via a microtubule segment 73. The outlet 71 is in fluid communication with the lower left vacuum aperture 65 via a microtubule segment 75 that is further in fluid communication with the segment 73 at the outlet 71. Each of the segments 73, 75 extend through the apertures 63, 65 to the central aperture 67, where they are in fluid communication with the central aperture 67.

The central aperture 67 and the right central aperture 69 are in fluid communication via both of an upper microtubule segment 77 and a lower microtubule segment 79. As shown in FIG. 5, the alternative middle layer 59 and its microtubule segments 73, 75, 77, 79 form two marquise shapes 81, 83. The first marquise shape 81 is generally more elongated and narrower than the second marquise shape 83.

In operation, when the vacuum source 3 is activated, a vacuum effect is provided at the outlet 71 that causes the various vacuum apertures 61 to suck through the top layer 19 any fluid that has spilled over the suction attachment 9. Via the system of microtubules 72, the excess fluid is sucked through the top layer 19, into the various vacuum apertures 61 and through the outlet 71 to the vacuum source 3, where it is disposed of.

In alternative embodiments not illustrated, the apertures 21, 61 of the middle layers 13, 59 may take on a nearly limitless number of configurations. Microtubule systems associated with different aperture arrangements may thus also take on a nearly limitless number of configurations. In those alternative embodiments, the apertures still should be in fluid communication with the vacuum source 3 via an outlet (and adapter 7, hose member 3 in the illustrated embodiments) so that when the vacuum source 3 is activated, suction is provided via the vacuum apertures and the microtubule system.

As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A suction device for reducing blood pooling in a surgical site, the suction device comprising:
a vacuum source; and
a suction attachment, the suction attachment including:
a bottom absorptive layer including each of a top surface and a bottom surface;
a middle layer including each of a top surface and a bottom surface, wherein the bottom surface of the middle layer is adhered to the top surface of the bottom layer; and
a top absorptive layer including each of a top surface and a bottom surface, wherein the bottom surface of the top layer is adhered to the top surface of the middle layer;
wherein the middle layer includes a plurality of vacuum apertures on its top surface connected to one another via a microtubule system; and
wherein the plurality of vacuum apertures are in fluid communication with the vacuum source via the microtubule system, such that when the vacuum source is activated, suction is provided via the plurality of vacuum apertures through the top layer.

2. The suction device of claim 1, wherein the bottom layer is made of cotton.

3. The suction device of claim 1, wherein the top layer is made of cotton.

4. The suction device of claim 1, wherein the middle layer is made of hydrophobic Polydimethylsiloxane.

5. The suction device of claim 1, wherein the top surface of the middle layer includes five vacuum apertures.

6. The suction device of claim 1, wherein the middle layer includes an outlet that is in fluid communication with each of the microtubule system and the vacuum source.

7. The suction device of claim 1, wherein the microtubule system is shaped as two parallelograms that are mirror images of one another.

8. The suction device of claim 1, wherein the top surface of the middle layer includes six vacuum apertures.

9. The suction device of claim 1, wherein the microtubule system is shaped as two marquise shapes.

10. The suction device of claim 1, wherein the top layer includes a radioactive tag.

11. A suction attachment for reducing blood pooling in a surgical site, the suction attachment comprising:
a bottom absorptive layer including each of a top surface and a bottom surface;
a middle layer including each of a top surface and a bottom surface, wherein the bottom surface of the middle layer is adhered to the top surface of the bottom layer; and
a top absorptive layer including each of a top surface and a bottom surface, wherein the bottom surface of the top layer is adhered to the top surface of the middle layer; and
wherein the middle layer includes a plurality of vacuum apertures on its top surface connected to one another via a microtubule system.

12. The suction attachment of claim 11, wherein the bottom layer is made of cotton.

13. The suction attachment of claim 11, wherein the top layer is made of cotton.

14. The suction attachment of claim 11, wherein the middle layer is made of hydrophobic Polydimethylsiloxane.

15. The suction attachment of claim 11, wherein the top surface of the middle layer includes five vacuum apertures.

16. The suction attachment of claim 11, wherein the middle layer includes an outlet that is in fluid communication with the microtubule system.

17. The suction attachment of claim 11, wherein the microtubule system is shaped as two parallelograms that are mirror images of one another.

18. The suction attachment of claim 11, wherein the top surface of the middle layer includes six vacuum apertures.

19. The suction attachment of claim 11, wherein the microtubule system is shaped as two marquise shapes.

20. The suction attachment of claim 11, wherein the top layer includes a radioactive tag.
